(19) United States
(12) Patent Application Publication
(10) Pub. No.: US 2008/0216841 A1
(43) Pub. Date: Sep. 11, 2008

(54) Nasal cannula

(76) Inventors: Beverly S. Grimes, Boxford, MA (US); Donald P. Ricklefs, Boxford, MA (US)

Correspondence Address: MCDERMOTT WILL & EMERY LLP, 28 STATE STREET, BOSTON, MA, 02109-1775, US

(21) Appl. No.: 11/715,614

(22) Filed: Mar. 8, 2007

(51) Int. Cl. A61M 16/00

(52) U.S. Cl. 128/207.18

(57) ABSTRACT

Described herein is a respiratory apparatus for delivering and detecting gases in a patient. The apparatus includes a first cannula having a distal end adapted to be received at a first depth in a nasal passage of the patient, and a second cannula having a distal end adapted to be received at a second depth in the nasal passage, wherein the distal end of the first cannula can receive a proximal end of the second cannula. The second cannula circumventing any obstruction of the nasal passage. Additionally, the apparatus has a septum which can have a membrane thus facilitating the delivery and detection of gases simultaneously.

### BACKGROUND OF THE INVENTION

While a nasal cannula can be highly effective in delivering oxygen and monitoring expired gas, it can be problematic under certain circumstances. For example, a split nasal cannula can only be used when both nasal passages are clear. When either nasal passage is closed or even partially obstructed, either oxygen delivery or carbon dioxide monitoring is compromised.

Another area of concern is a deviated nasal septum. Deviated nasal septums can preclude or restrict the use of a nasal cannula for delivery of oxygen. Currently, a nasal cannula cannot easily be employed for an individual with a deviated septum or some other condition that restrict the nasal passage.

### SUMMARY

The present invention pertains to an improved respiratory apparatus that comprises a nasal cannula that can be used to circumvent nasal obstructions. Such nasal obstructions include a deviated septum, also included, are nasal surgical procedures which require the cannula to bypass the surgical field.

The invention provides an improved respiratory apparatus for delivering oxygen and other gases via utilization of the nasal passage. Additionally, the apparatus of the instant invention can be used to detect gases expelled by an individual such as carbon dioxide.

The present apparatus includes a first cannula having a distal end adapted to be received at a first depth in, for delivering (receiving) a gas into (or from), a nasal passage of the patient, and a second cannula having a distal end adapted to be received at a second depth in the nasal passage, wherein the distal end of the first cannula can receive a proximal end of the second cannula. The second cannula circumventing any obstruction of the nasal passage.

A method of making an apparatus for delivering gas (e.g., oxygen) or detecting gas (e.g., carbon dioxide) involves modifying an apparatus having a first cannula. This first cannula is designed to deliver or detect fluid to or from a patient. This first cannula is connected or affixed to a second cannula. The method of delivering or detecting gas to/from a patient includes inserting to a first depth the distal end of the first cannula in a nostril of the patient which will permit the affixed second cannula to circumvent any nasal obstruction such as a deviated septum. The second cannula has a longitudinal dimension such that it bypasses the nasal septum.

The present invention provides improved elements and arrangements thereof, for the purposes described, which are inexpensive, dependable and effective in accomplishing intended purposes of the invention. Other features and advantages of the present invention will become apparent from the following description of the embodiments which refers to the accompanying drawings.

### BRIEF DESCRIPTION OF THE DRAWINGS

FIG. 1 depicts a respiratory apparatus of the present invention.

FIG. 2 depicts a septum of the present invention.

### DETAILED DESCRIPTION

The present invention relates to the delivery of a gas, fluid or alike to a patient using the nasal passage. Specifically, the current invention pertains to an improved nasal cannula that can be employed to circumvent obstructions of the nasal passage. Such obstructions include, but are not limited to, a deviated septum.

In one embodiment, an improved cannula apparatus for delivering oxygen and other gases via utilization of the nasal passage is described. The apparatus for delivering gas to a patient includes a first cannula having a distal end adapted to be received at a first depth in, for delivering a gas into, a nostril of the patient, and a second cannula having a distal end adapted to be received at a second depth in the nostril, wherein the distal end of the first cannula can receive a proximal end of the second cannula. The second cannula circumventing any obstruction of the nasal passage. The method of making an apparatus for delivering gas (e.g., oxygen) involves modifying an apparatus, including a first cannula connected for delivering fluid to a nasal cannula, and a second cannula connected to the first nasal cannula. The method of delivering gas to a patient includes inserting to a first depth a distal end of a first cannula in a nostril of the patient which will permit the affixed second cannula to circumvent any nasal obstruction such as a deviated septum.

In another embodiment, the present apparatus can be used to detect gases from a patient. Such gases include carbon dioxide.

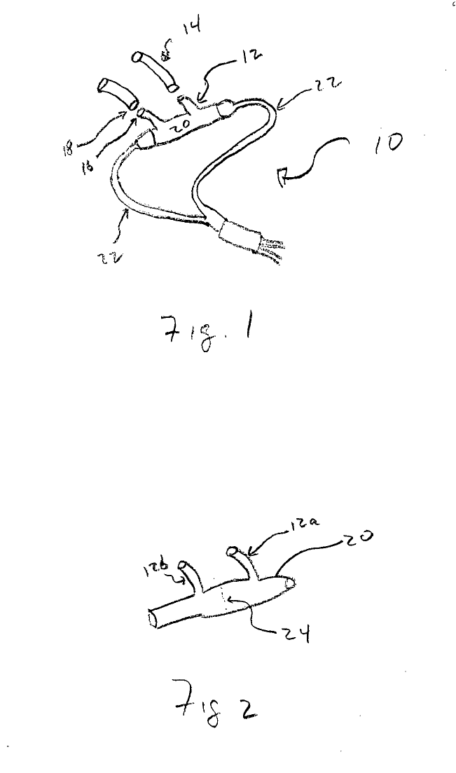

Referring to FIG. 1, a typical nasal apparatus 10 is shown (absent element 14) for the delivery of a, e.g., a gas (such as oxygen) to a patient. This apparatus is minimally invasive and can usually be tolerated by the patient. The apparatus 10 comprises a first cannula 12 which penetrates the nasal passage anywhere from about 1 mm to about 1.5 cm. This first cannula arises from septum 20. The septum 20 is in fluid connect with a source of gas and/or a detector via one or more fluid conduits 22.

When encountering a blockage or narrowed nasal passage it can be difficult to deliver and inhalant such as oxygen. (By inhalant it is to be understood that both gases and liquids are included.) This can be due to, for example, a deviated septum. By essentially elongating the cannula in order to bypass the obstruction, delivery of the inhalant can be effected. Referring to FIG. 1 again, a second cannula 14 can be affixed to the first cannula 12, wherein the first and second cannuli are in fluid communication. The second cannula can be any suitable dimension that facilitates delivery of an inhalant to a patient bypassing any obstruction in the nasal canal. For example, the second cannula 14 can range from about 5 mm to about 5 cm or more.

The second cannula can be affixed to the first cannula by any means well known to those skilled in the art. For example, the second cannula can be affixed to the first cannula by friction. A suitable adhesive can be applied so that the second cannula is permantly affixed to the first cannula.

Materials suitable for the second cannula is that which is well known by the skilled artisan. Preferably, the second cannula will be flexible yet non-collapsible, such as the material currently used to manufacture nasal cannula.

Referring to FIG. 2, a septum 20 of the present invention can comprise a membrane 24 such that the first cannulas 12a and 12b can have diverse functions. For example, cannula 12a can be used to deliver a gas such as oxygen while cannula 12b can be employed to detect a gas such as carbon monoxide. (It should be obvious to one skilled in the art that there is no significance to which first cannula is used for a particular purpose.) The membrane 24 can be comprised of any suitable material known to those skilled in the art such as a plastic. This membrane 24 facilitates the delivery and detection of gases simultaneously.

The respiratory apparatus of the present invention to be secures about a patient's head by means well known to those skilled in the art.

While this invention has been particularly shown and described with references to specific embodiments, it will be understood by those skilled in the art that various changes in form and details may be made therein without departing from the spirit and scope of the invention as defined by the appended claims.

What is claimed is:

1. A respiratory apparatus comprising:
(a) a septum, wherein said septum has one or more first cannuli;
(b) one or more second cannuli in fluid communication with said one or more first cannuli; and
(c) one or more fluid conduits in communication with said septum.

2. The respiratory apparatus of claim 1, wherein said septum has a membrane.

3. The respiratory apparatus of claim 1, wherein said first cannula ranges from about 1 mm to about 1.5 cm.

4. The respiratory apparatus of claim 1, wherein said second cannula ranges from about 5 mm to about 5 cm.

5. The respiratory apparatus of claim 1, wherein said apparatus has at least two fluid conduits, and wherein one of said fluid conduits is used to detect a gas and the other fluid conduit is used to deliver a gas.
